(19) United States
(12) Patent Application Publication
(10) Pub. No.: US 2008/0206803 A1
(43) Pub. Date: Aug. 28, 2008

(54) Metabolic genes and related methods and compositions

(75) Inventor: Justin Gallivan, Atlanta, GA (US)

Correspondence Address: ROPES & GRAY LLP, PATENT DOCKETING 39/41, ONE INTERNATIONAL PLACE, BOSTON, MA, 02110-2624, US

(73) Assignee: California Institute of Technology, Pasadena, CA (US)

(21) Appl. No.: 12/012,781

(22) Filed: Feb. 5, 2008

Related U.S. Application Data
(63) Continuation of application No. 10/260,994, filed on Sep. 30, 2002, now abandoned.
(60) Provisional application No. 60/325,636, filed on Sep. 28, 2001.

(51) Int. Cl. C12Q 1/02; C12N 15/00; C12N 15/87; C12N 15/74

(52) U.S. Cl. 435/29; 435/320.1; 435/325; 435/455; 435/468; 435/471

(57) ABSTRACT

Certain aspects of the invention provide nucleic acid constructs that can be used to cause a cell to be dependent on a particular enzymatic activity or on the presence of a particular small molecule. Certain aspects of the invention also provide methods for cloning genes involved in the synthesis, modification or degradation of a given molecule and for the directed evolution of proteins that perform a specified enzymatic function. Certain methods of the invention can be used to isolate the genes responsible for directing the biosynthesis, modification or degradation of a particular target molecule and to isolate polypeptide variants having new or improved enzymatic activity.

### RELATED APPLICATIONS

This Application is a continuation application of U.S. Ser. No. 10/260,994, filed on Sep. 30, 2002, which claims the benefit of the filing date of U.S. Provisional Application No. 60/325,636, entitled “Methods for the Cloning of Biosynthesis Genes and the Directed Evolution of Proteins,” by Justin Gallivan and filed Sep. 28, 2001. The teachings of the referenced applications are incorporated by reference herein in their entirety.

### BACKGROUND OF THE INVENTION

Enzymes catalyze a wide range of useful chemical reactions. In many instances, the use of an enzyme to synthesize a compound provides a variety of advantages over the traditional methods of synthetic organic chemistry. For example, enzymatically catalyzed reactions generally occur at a moderate temperature and pH and use environmentally benign reactants. In addition, enzymatic reactions are often stereospecific, thus avoiding any need for the laborious process of separating stereoisomers. Additionally, enzymes can perform their functions from within a cell or in a purified form. A nucleic acid encoding an enzyme of interest may be inserted into a cell so that the cell produces the enzyme, perhaps enhancing a function of those cells. For example, organisms such as plants and bacteria are widely used as natural tools for degrading certain hazardous waste compounds, and, if appropriate enzymes were available, these organisms could be modified to produce additional waste-degrading enzymes to achieve a more efficient environmental detoxification.

Although the advantages of using enzymes for synthetic and decomposition reactions are well known, it is often difficult for scientists and engineers to obtain an enzyme that performs a desired chemical reaction. Rational design methods for generating artificial enzymes have been slow to develop, and the use of enzymes for synthetic reactions has tended to be limited to those enzymes that are found in natural sources. In the last decade, methods have been developed for increasing the diversity of available enzymes. For example, it is now possible to “shuffle”, or mix and match the sequences of, a set of related genes to generate a highly diversified pool of genes encoding novel enzymes. Additionally, it is now understood that a vast number of organisms that produce potentially useful enzymes are unculturable, but the genes from unculturable organisms can now be accessed by isolating nucleic acids directly from environmental samples to generate so-called environmental DNA (or eDNA). These libraries and eDNA pools can be screened to identify genes encoding enzymes with desirable properties, and these desirable enzymes may be subjected to rounds of diversification and screening to further optimize the enzymes. This process of generating and optimizing useful enzymes is sometimes called directed evolution.

A major limitation in the field of directed evolution is the need to devise systems that allow for the selection of cells that are dependent on the desired enzymatic activity. The process of screening large libraries of genes encoding a wide range of enzymes is labor intensive. In effect, the ability of scientists to generate variation in enzymatic capabilities has outpaced the ability of scientists to sift through the large numbers of variants to find the desired enzymes. Likewise, it has been difficult to rapidly isolate the genes involved in the biosynthesis, degradation or modification of a particular natural product.

Accordingly, there exists a need for new tools to discover genes involved in the biosynthesis, modification and degradation of various natural products.

### SUMMARY OF THE INVENTION

In certain aspects, the invention provides nucleic acid constructs, systems and methods for the control of gene expression (mRNA translation) in cells and for the detection of target molecules in cells. In certain embodiments, methods of the invention may be used to identify nucleic acids encoding polypeptides or ribonucleic acids (RNAs) involved in the biosynthesis, degradation or other modification of a target molecule. In certain embodiments, methods of the invention may be used to generate a cell-based biosensor for a target molecule. In certain preferred embodiments, the invention provides cells that are dependent on a target molecule for growth or viability, and such cells may be used, for example, in the rapid selection of enzymes having desirable catalytic activities.

In certain aspects, the invention provides nucleic acid constructs, and systems of nucleic acid constructs, comprising an aptamer region and a coding region. An aptamer is a nucleic acid sequence that interacts with a target molecule of interest to form an aptamer:target molecule complex. The coding region can encode a polypeptide and/or an RNA molecule. The nucleic acid constructs, and systems of nucleic acid constructs, are designed so that the formation of an aptamer:target molecule complex regulates the production of the product encoded by the coding region. Constructs and systems of this aspect of the invention permit controlled expression of a coding region in cells and may be used, for example, in methods for detecting a target molecule in a cell. In certain preferred constructs and systems, the coding region encodes a toxin, and the production of the toxin is inhibited by the aptamer:target molecule complex. In further preferred embodiments, the invention provides cells comprising said preferred construct or system, such that the cells are dependent on the presence of the target molecule to inhibit production of the toxin and, therefore, to allow growth and/or viability of the cells.

In certain embodiments, the invention provides nucleic acid constructs wherein the aptamer and the coding region are transcribed as a single RNA transcript, and binding of the target molecule to the aptamer causes a decrease in the production of the product encoded by the coding region. In further embodiments, the invention provides systems of nucleic acid constructs wherein the aptamer:target molecule complex regulates expression of the coding region indirectly. For example, the aptamer:target molecule may regulate production of a regulatory factor that in turn regulates the expression of a coding region.

In certain embodiments, a nucleic acid construct of the invention further comprises a promoter that drives transcription of the aptamer and/or coding region. Optionally, the promoter is a conditional promoter, meaning that the promoter drives transcription under certain conditions but not others. A nucleic acid construct of the invention may further comprise a resistance gene. A resistance gene allows the selection of cells that comprise the nucleic acid construct, thereby preventing loss of the nucleic acid construct from the cell.

In certain embodiments, the invention provides vectors comprising one or more nucleic acid constructs of the invention. Vectors may be designed for use in one or more host cell types, including eukaryotic cell types and bacterial cell types. In certain embodiments, a vector is designed to integrate into the genome of a host cell. In certain embodiments, a vector is designed to replicate as a free episome in the host cell, and such vectors will generally include appropriate sequences for replication in a particular host cell.

In certain embodiments, the invention provides cells comprising one or more nucleic acid constructs or systems described herein. Nucleic acid constructs, and systems of nucleic acid constructs, described herein may be used to generate cell lines that evince a change in the production of a detectable signal in the presence of a target molecule. For example, constructs described herein may be used to generate cells that are dependent upon a particular target molecule for growth or viability. As another example, constructs described herein may also be used to generate cells that produce (or stop production of) a detectable signal (such as a bioluminescent, fluorescent or colored compound) in the presence or absence of a target molecule. The various cell types that can be generated may be used for a variety of purposes, including the identification of nucleic acids encoding proteins or RNAs involved in the metabolism of a compound of interest, the engineering or optimization of proteins or RNAs involved in metabolism of a compound of interest, and construction of biosensors.

In certain embodiments, cells of the invention further comprise an exogenous nucleic acid. Such cells may be used, for example, to assess the effects of the exogenous nucleic acid on the target molecule. An exogenous nucleic acid encoding a polypeptide or RNA involved in the synthesis, degradation or other modification of the target molecule is expected to alter the amount of target molecule present in the cell, thereby altering the expression of a coding sequence that is regulated by an aptamer:target molecule complex. Exogenous nucleic acids may be essentially any nucleic acid of interest, including a nucleic acid selected (purposefully or randomly) from a nucleic acid library. In certain preferred embodiments, an exogenous nucleic acid is a sample from a library that contains sequences encoding a diverse set of enzymes having varied catalytic properties. The exogenous nucleic acid may be provided separately from the nucleic acid construct(s) comprising the aptamer and the coding sequence, or the exogenous nucleic acid may be provided as part of a construct comprising the aptamer and/or coding sequence.

In certain aspects, the invention provides methods for cloning or assisting in cloning a gene involved in the metabolism of a target molecule. Methods according to this aspect comprise culturing a test cell that comprises: (a) a first nucleic acid construct comprising an exogenous nucleic acid and (b) a second nucleic acid construct comprising a coding region encoding an aptamer that binds a target molecule to form an aptamer:target molecule complex and a coding region encoding a reporter product, wherein the aptamer:target molecule complex regulates production of the reporter product, and observing an effect of the expression of the exogenous nucleic acid on the production of the reporter product, wherein an exogenous nucleic acid that affects the production of the reporter product is a gene involved in the metabolism of the target molecule. Optionally, observing an effect of the expression of the exogenous nucleic acid on the production of the reporter product comprises a comparison to an appropriate control cell. Appropriate control cells are, for example, identical to the test cells except that they lack the exogenous nucleic acid or do not express the exogenous nucleic acid. Results obtained from test cells can be compared to results obtained from control cells cultured at the same time or with a previously established control. Optionally, the coding region encoding the aptamer and the coding region encoding the reporter product are positioned such that the aptamer directly regulates the production of the reporter product. Optionally, the coding region encoding the aptamer and the coding region encoding the aptamer are arranged such that the aptamer indirectly regulates the production of the reporter product. Accordingly, the coding region encoding the aptamer and the coding region encoding the reporter product may be positioned so as to be transcribed as a single mRNA, or these two coding regions may be separated onto, for example, different vectors or different positions in the chromosome of a cell.

Certain embodiments of the disclosed methods for cloning genes involved in the synthesis, degradation or other modification of a target molecule are of particular use when a natural product has been discovered, but the metabolic machinery is unknown. In certain embodiments a method of the invention is as follows: a host cell is transformed with a nucleic acid construct having an aptamer region that can interact with such a target molecule. The aptamer region regulates the expression of a coding sequence encoding a reporter product. Optionally, the aptamer region is operably linked to the reporter sequence region. The reporter region sequence can encode a polypeptide or RNA molecule, the production of which can be detected either directly (for example as an fluorescent signal, or through influence on the growth, behavior, morphology, etc.) or indirectly, for example through the enzymatic conversion of a substrate to form a color change or luminescence, of the host cell. In a preferred embodiment, the reporter region encodes a toxic polypeptide or RNA molecule. In such an embodiment, the host cell's growth is impaired or the host cell dies in the absence of the target molecule.

In certain aspects, the invention provides methods for identifying or assisting in identifying a variant of a protein having an altered activity, such as a binding activity or catalytic activity, with respect to a target molecule. In certain embodiments, such a method comprises culturing a cell comprising: (a) a first nucleic acid construct comprising a coding region encoding an aptamer that binds a target molecule to form an aptamer:target molecule complex and a coding region encoding a reporter product, wherein the aptamer:target molecule complex regulates production of the reporter product, and (b) a second nucleic acid construct comprising a coding region encoding a variant polypeptide, wherein the culture conditions are appropriate for expression of the variant polypeptide, and observing the production of the reporter product, wherein a variant polypeptide that causes a change in the production of a reporter product as compared to a suitable control cell is a variant having an altered activity with respect to the target molecule. Appropriate control cells, for example, identical to the test cells except that they comprise a coding region encoding a control polypeptide instead of the variant polypeptide. Results obtained from test cells can be compared to results obtained from control cells cultured at the same time or with a previously established control. Variants of a protein may include proteins with insertion mutations, deletion mutations, point mutations, a shuffling of sequence blocks and mixtures of the foregoing.

In certain embodiments, the invention provides a method for detecting the presence of a target molecule, the method comprising culturing a cell in an environment suspected of containing the target molecule, the cell comprising an aptamer coding sequence and a toxin coding sequence, wherein the aptamer regulates production of the toxin in response to a target molecule, and observing the production of the toxin. An environment that causes a change in the production of the toxin is an environment that contains the target molecule.

### BRIEF DESCRIPTION OF THE DRAWINGS

FIG. 1 shows three possible growth conditions for an E. coli cell expressing an aptamer/toxic protein construct. The black square represents the target molecule that binds to the aptamer. The black circle represents a vector encoding sequences that cause production of the target molecule.

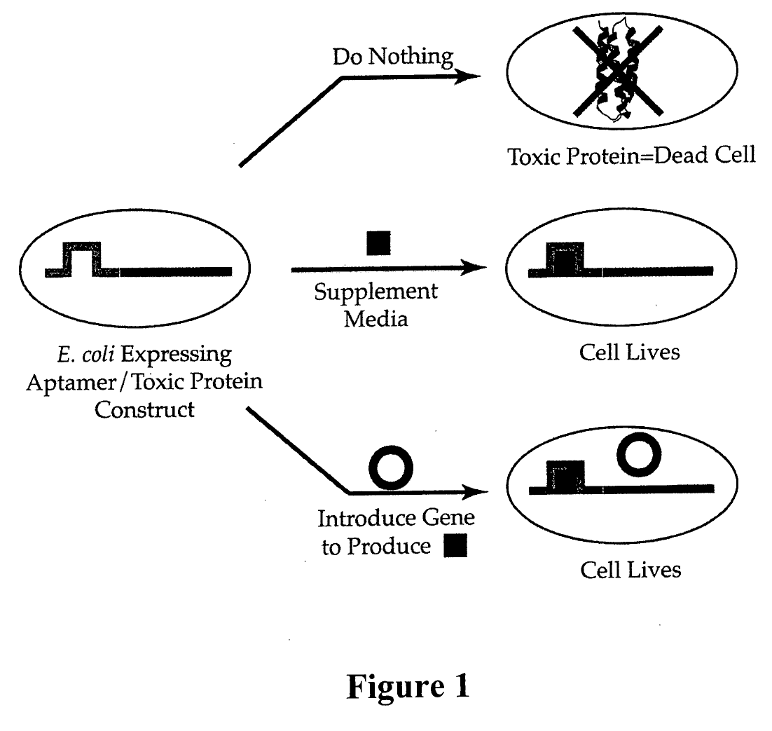

FIG. 2 is a schematic representation of a vector comprising an inducible promoter, an aptamer that regulates expression of a toxic gene (barnase) and an antibiotic resistance cassette. FIG. 2 also shows a control experiment to verify that a cell carrying the vector is dependent on the target molecule (caffeine, in this instance) for survival. An E. coli cell carrying the vector is represented by a large black oval (the schematic cell outline) with a gray-shaded circle (the vector) inside.

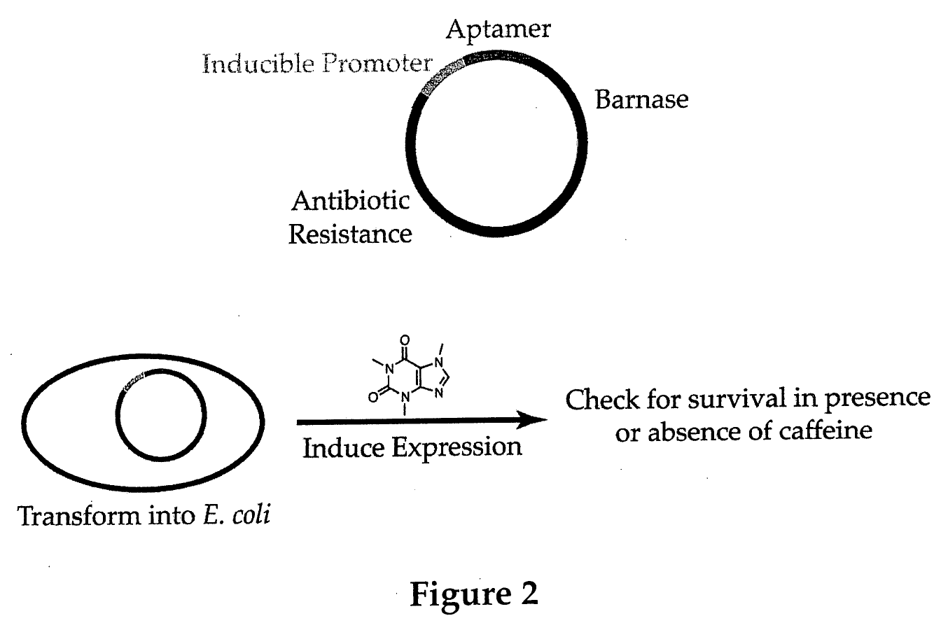

FIG. 3 is a schematic representation of a method for using a cell line that is dependent on caffeine for viability to isolate nucleic acids involved in caffeine metabolism from a coffee plant cDNA library.

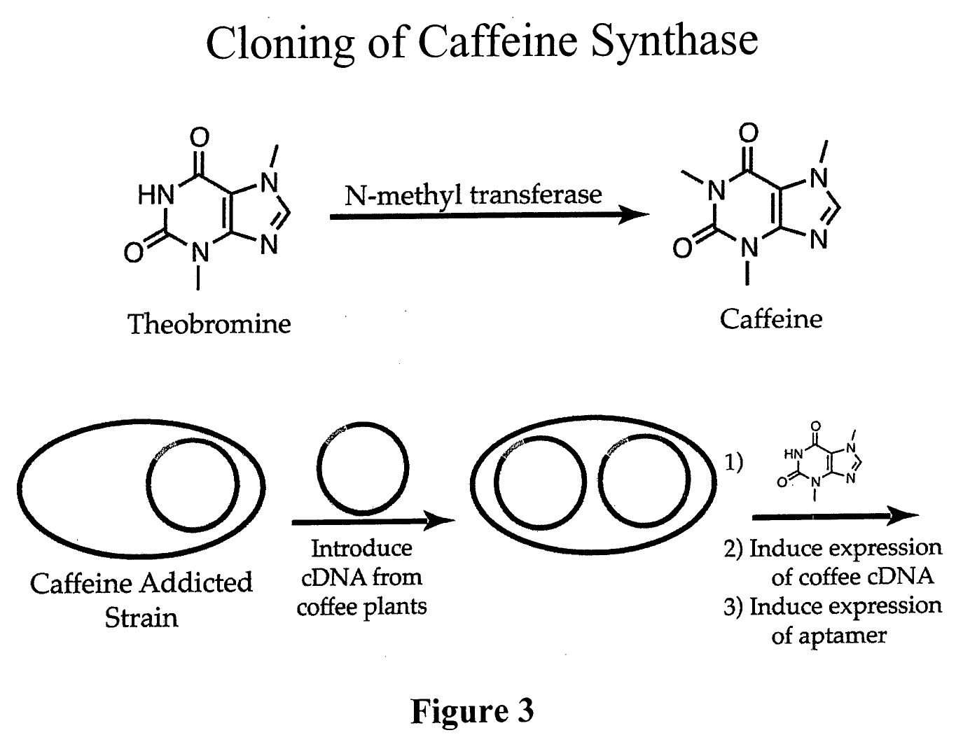

FIG. 4 is a schematic representation of a method for using a cell line that is dependent on theophylline for viability to isolate nucleic acids involved in theophylline metabolism from a coffee plant cDNA library.

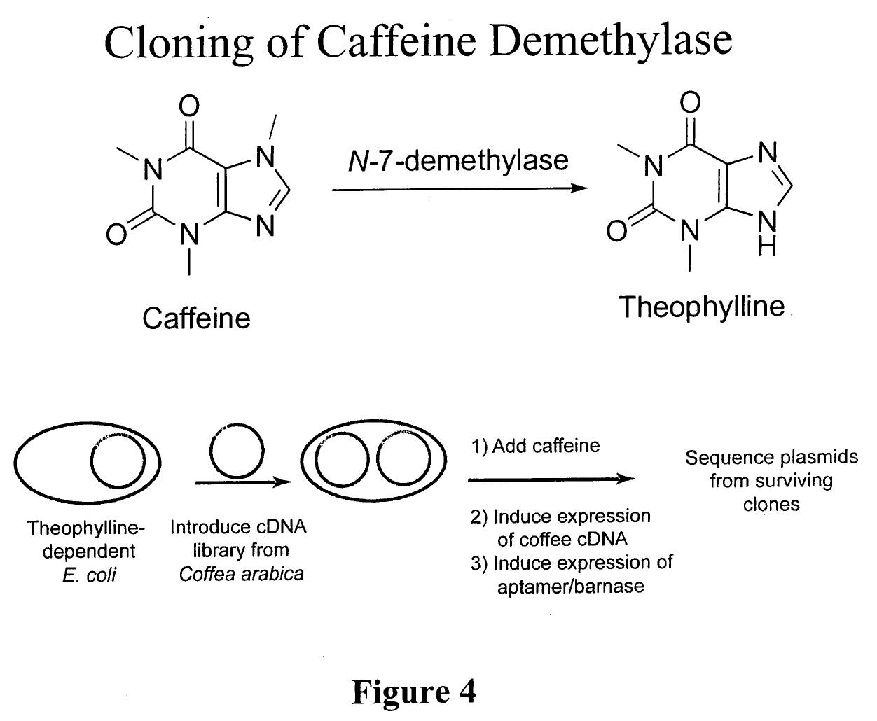

### DETAILED DESCRIPTION

### 1. Definitions

For convenience, certain terms employed in the specification, examples, and appended claims are presented here. Unless defined otherwise, all technical and scientific terms used herein have the same meaning as commonly understood by one of ordinary skill in the art to which this invention belongs.

The term “aptamer” includes any nucleic acid sequence that is capable of specifically interacting with a target molecule. An aptamer may be a naturally occurring nucleic acid sequence or a nucleic acid sequence that is not naturally occurring. Aptamers may be any type of nucleic acid (e.g. DNA, RNA or nucleic acid analogs) and may be single-stranded or double-stranded. In certain specific embodiments described herein, aptamers are a single-stranded RNA.

An “aptamer:target molecule complex” is a complex comprising an aptamer and the target molecule with which it interacts. The aptamer and the target molecule need not be directly bound to each other.

A “coding region” includes polynucleotide regions that, when present in a DNA form, can be expressed as an RNA molecule. The coding region may encode, for example, a polypeptide produced through translation of the RNA. A coding region may also encode an RNA that is not translated into a polypeptide, such as an RNA aptamer, a ribosomal RNA or other biologically active RNA molecule.

The term “derived from” as used herein in reference to a nucleic acid means that at least a portion of the nucleic acid (e.g. gene, gene portion, regulatory element, polypeptide) is also present in (or was copied from) the biological source that the nucleic acid was derived from. The derived nucleic acid may be constructed in any way that provides the desired sequence, including the derivative portion. For example, nucleic acid may be obtained directly from a biological source, using restriction enzymes or other tools of molecular biology, or by amplifying from a biological source (e.g., by polymerase chain reaction), or by a technique such as chemical synthesis. While in many instances a nucleic acid derived from a biological source is not directly obtained from the source, its sequence and/or characteristics are substantially the same as a portion of sequence from the biological source.

The term “including” is used herein to mean, and is used interchangeably with, the phrase “including but not limited to”.

The term “nucleic acid” refers to polynucleotides such as deoxyribonucleic acid (DNA), and, where appropriate, ribonucleic acid (RNA). The term also includes analogs of either RNA or DNA made from nucleotide analogs, and, as applicable to the embodiment being described, single (sense or antisense) and double-stranded polynucleotides.

The term “nucleic acid construct” is used herein to mean any nucleic acid comprising sequences which are not adjacent in nature. A nucleic acid construct may be generated in vitro, for example by using the methods of molecular biology, or in vivo, for example by insertion of a nucleic acid at a novel chromosomal location by homologous or non-homologous recombination. One or more nucleic acid constructs may be present in a single vector or chromosome.

A “nucleic acid library” is any collection of a plurality of nucleic acid species (nucleic acids having different sequences) isolated from a source, such as a culture of a particular cell type or a particular environmental sample. The nucleic acids of a library are generally situated in vectors, with one nucleic acid species (or “insert”) per vector.

The terms “polypeptide” and “protein” are used interchangeably herein.

The term “promoter” is used herein to refer to any nucleic acid that provides sufficient cis-acting nucleic acid regulatory elements to support the initiation of transcription of an operably linked nucleic acid in the appropriate conditions. Appropriate conditions may include the presence or activation of appropriate trans-acting factors, such as an RNA polymerase, a sigma factor or a transcription factor. Appropriate conditions may also include the absence or inactivation of negative regulatory factors, such as repressors. Appropriate conditions may further include chemical and physical conditions such as pH and temperature that are compatible with promoter function. Exemplary regulatory elements that may be part of a promoter include sigma factor binding sites (generally in bacterial and bacteriophage promoters), transcription factor binding sites, small molecule binding sites, repressor binding sites, etc. A promoter may be affected by one or more cis-acting or trans-acting element that is external to the promoter. Many promoters are “conditional” or “regulated” meaning that the degree to which the promoter supports the initiation of transcription is affected by one or more conditions inside or outside the cell.

A “reporter product” is any detectable substance that is produced by transcription, and optionally translation, of a nucleic acid. Generally, reporter products are selected for ease of detection, as in the case of fluorescent proteins and proteins that degrade or produce fluorescent or chromogenic compounds. However, a variety of biomolecules are detectable by traditional antibody- or nucleic acid probe-based techniques, and therefore a large array of reporter products are available. The presence or absence, and optionally the quantitative amount, of a reporter product may be observed by a variety of methods, including direct detection of the reporter product (e.g. detecting amounts of protein or nucleic acid) or detection of a product of the reporter product (e.g. detecting a result of enzymatic activity, emitted light, etc.).

As used herein, the term “small molecule” refers to a molecule having a molecular weight of less than about 5,000, more preferably a molecular weight of less than about 2,000 and even more preferably a molecular weight of less than about 1,000.

A “target molecule” is any compound of interest, including polypeptides, small molecules, ions, large organic molecules (such as various polymers and copolymers), as well as complexes comprising one or more molecular species.

A “toxin”, as the term is used herein, includes any molecule or collection of molecules that inhibit the growth of a host cell or cause the death of a host cell. The term toxin is intended to encompass toxins that directly inhibit cell growth or cause cell death, and toxins that act indirectly by, for example, catalyzing the synthesis of a direct toxin.

The terms “transform” and “transfect” are used interchangeably herein and include any process for causing a cell, including eukaryotic and prokaryotic cells, to take up an exogenous nucleic acid. Examples of transformation or transfection techniques include electroporation, calcium chloride transformation, virus-mediated transformation and lipid-mediated transformation.

The term “vector” refers to a nucleic acid molecule capable of transporting another nucleic acid to which it has been linked. One type of vector is an episome, i.e., a nucleic acid capable of extra-chromosomal replication, such as a plasmid. Another type of vector is an integrative vector that is designed to recombine with the genetic material of a host cell. Vectors may be both autonomously replicating and integrative, and the properties of a vector may differ depending on the cellular context (a vector may be autonomously replicating in one host cell type and purely integrative in another host cell type). Vectors designed to express coding sequences are referred to herein as “expression vectors”.

### 2. Nucleic Acid Constructs

In certain aspects, the invention relates to nucleic acid constructs and systems comprising an aptamer region and a coding region, wherein the aptamer region encodes an aptamer and wherein the coding region encodes a reporter or regulatory product. In certain preferred embodiments, the reporter product is a toxin.

Aptamers for use in various embodiments of the invention include any nucleic acid sequence that interacts with a target molecule. The interaction may involve direct or indirect binding, and will preferably be a specific interaction. An aptamer may be a naturally occurring nucleic acid sequence or a nucleic acid sequence that is generated in vitro. Many sequences generated in vitro will, by chance or otherwise, also be found in nature. While the technology is available to generate aptamers of any type of nucleic acid, including single- and double-stranded nucleic acids, DNAs, RNAs and polymers comprising nucleic acid analogs, many embodiments described herein preferably employ a single-stranded RNA aptamer.

In certain preferred embodiments, the aptamer is any RNA sequence that specifically interacts with a target molecule. RNA aptamer sequences are known for many target molecules, and it is possible to generate RNA sequences, known as aptamers, that bind small molecules with high affinity and specificity (Wilson, D.; Szostak, J. Annu. Rev. Biochem. 1999, 68, 611-647). For example, methods are well established for generating aptamers that bind to antibiotics. See, e.g., Wallace S T, Schroeder R “In vitro selection and characterization of RNAs with high affinity to antibiotics” RNA-Ligand Interactions, Part B; Methods In Enzymology 318:214-229, 2000. Such techniques have been used, for example to select an aptamer to Kanamycin B (Kwon M, Chun S M, Jeong S, Yu J (2001) “In vitro selection of RNA against kanamycin B,” Molecules and Cells 11: (3) 303-311).

Aptamer sequences also can be generated according to methods known to one of skill in the art, including, for example, the SELEX method described in the following references: U.S. Pat. Nos. 5,475,096; 5,595,877; 5,670,637; 5,696,249; 5,773,598; 5,817,785. The SELEX method is summarized below. A pool of diverse DNA molecules is chemically synthesized, such that a randomized or otherwise variable sequence is flanked by constant sequences. A DNA molecule having a variable sequence flanked by constant sequences may be generated, for example, by programming a DNA synthesizer to add discrete nucleotides (e.g. an A, T, G or C) to the growing polynucleotides during synthesis of constant regions and to add mixtures of nucleotides (e.g. an A/T mixture, an A/T/G mixture or an A/T/G/C mixture) to the growing polynucleotides during synthesis of the variable region. When an A/T mixture is added to growing polynucleotides, the result will be a mixture of polynucleotides, some having an A at the newly synthesized position, and some having a T at the newly synthesized position. One of the constant regions generally comprises an RNA polymerase promoter (e.g. a T7 RNA polymerase promoter) positioned to allow transcription of the variable sequence and, optionally, portions of or all of one or both of the flanking constant sequences. The RNA molecules are then partitioned according to a desired characteristic, such as the ability to bind to a target molecule. For example, a target molecule may be affixed to a resin and poured into a chromatography column. The RNA molecules are then passed over the column. Those that do not bind are discarded. RNAs that do bind the target molecule column may be eluted (e.g. with excess of the target molecule, or a guanidinium-HCl or urea solution). These binding RNAs are then converted back into DNA using reverse transcriptase, amplified by polymerase chain reaction (which may involve the use of primers that restore the RNA polymerase promoter, if necessary). The cycle may then be repeated progressively enriching for aptamers that have a potent affinity for the target molecule. In instances where it is desirable to obtain an aptamer that binds to a target molecule but does not bind to another compound (such as a structurally similar precursor molecule), additional selections may be performed to remove those aptamers that bind to the non-target molecule. For example, a column of aptamers bound to the target molecule may be flushed with the non-target molecule to remove aptamers with significant interaction with the non-target molecule. These methods are adaptable for generating single stranded or double stranded aptamers. (Thiesen H-J, Bach C. (1990) Nucleic Acids Res. 18:3203-09; Ellington A D, Szostak J W (1992) Nature 355:850-52). Using techniques such as SELEX, one of skill in the art can generate an aptamer sequence capable of interacting with a target molecule, and the degree of specificity of binding (i.e. lack of binding to other compounds) can also be selected.

Many natural sequences with specific binding properties are also known, and nucleic acids encoding such sequences may be used as aptamer coding sequences of the invention. For example, if the target molecule is coenzyme B12, the 5′untranslated region of the E. coli btuB gene may be used as an aptamer (Nahvi et al. 2002, Chemistry & Biology 9:1043-49). Other naturally occurring nucleic acids that bind possible target molecules are also known (see, for example, Miranda-Rios et al. 2001, Proc. Natl. Acad. Sci. USA 98:9736-41).

Aptamers suitable for use in the methods described herein may be selected empirically. In certain embodiments, a set of candidate aptamers may be screened by testing the candidates in vivo for their ability to regulate production of a reporter product in response to the presence of the target molecule. For example, many embodiments of the invention employ an aptamer that, when bound to target molecule, inhibits translation of an mRNA. While not wishing to be bound to any particular theory describing the mechanism by which an aptamer achieves this effect, it has been demonstrated that mRNA secondary structure in the 5′-untranslated region (5′-UTR) of a gene dramatically and predictably inhibits the translation of the mRNA downstream (see, for example, de Smit and van Duin 1994, J. Mol. Biol. 1994:144-50). Werstuck and Green have used the binding of a small molecule to an aptamer sequence in the 5′-UTR to control translation in eukaryotic cells (Werstuck, G.; Green, M. Science 1998, 282, 296-298). Accordingly, an aptamer for use in such embodiments may be selected for its ability to inhibit translation of an mRNA upon binding a target molecule. In certain preferred embodiments the aptamer is substantially devoid of secondary structure in the absence of the target molecule, and undergoes an increase in secondary structure formation in the presence of target molecule. In certain embodiments, the aptamer has intrinsic secondary structure that is further stabilized by binding of the target molecule. While the performance of an aptamer in the actual assay for which it is to be used, such as a translation inhibition assay, is of significant importance in selecting an aptamer, other properties may be used, alone or in combination, to identify or describe a suitable aptamer. For example, the affinity and/or specificity of the interaction between an aptamer and the target molecule may be measured, and such information may be useful for selecting or describing aptamers that are appropriate for a particular task.

As described above, it is possible to generate aptamers that vary in their binding affinities for the target molecule. The importance of using an aptamer with a high or low affinity for the target molecule will depend on the nature of the experiment to be conducted, and as discussed above, the affinity will often be of secondary importance to other properties, such as the secondary structure formed by the aptamer upon binding to the target molecule or the ability of the aptamer:target molecule complex to inhibit translation of an mRNA. The term low affinity is used herein to refer to aptamers having a dissociation constant (K_{D}) of 10⁻³M or greater. The term moderate affinity is used herein to refer to aptamers having a K_{D} of between 10⁻⁶M and 10⁻³M. The term high affinity is used herein to refer to aptamers having a K_{D} of less than 10⁻⁶M. Recognizing that, in certain embodiments, the aptamer will have several properties including the ability to adopt different secondary structures depending on conditions such as binding to a target molecule, and that these different properties may complicate how an aptamer behaves in a particular assay, an aptamer with a lower K_{D} will tend to provide a more sensitive assay, as it will bind the target molecule at lower concentrations, and accordingly the affinity of the aptamer may be selected depending on the desired sensitivity. In instances where it is desirable to distinguish between a background level of the target molecule and a higher level of the target molecule, a high affinity aptamer may tend to give a “noisy” signal by binding to the target molecule even at the lower background levels. In such instances an aptamer having low to moderate affinity may be preferable. A tandem series of aptamers may also be employed.

As described above, it is possible to generate aptamers having a range of different specificities with respect to the target molecule. Specificity, as the term is used herein, is defined relative to a particular non-target molecule. Specificity is herein defined as the ratio of the K_{D} of the aptamer for binding the target molecule to the K_{D} of the aptamer for binding a particular non-target molecule. For example, if the aptamer has a K_{D} of 10⁻⁶M for the target molecule and 10⁻⁵M for the non-target molecule, the specificity is 10 (10⁻⁶/10⁻⁶). The importance of using an aptamer with a high or low specificity for the target molecule relative to a particular non-target molecule will depend on the nature of the experiment to be conducted. In embodiments where the aptamer is used to regulate expression of a reporter product in response to the production of a metabolite (target molecule) by a cell, it will generally be preferable to employ an aptamer that has a specificity greater than 1 with respect to the most abundant precursor of the target molecule. In instances where the metabolite is further processed by the cell to give downstream metabolites, it may be preferable to employ an aptamer that has a specificity greater than 1 with respect to the most abundant downstream metabolites. In instances where the aptamer is to be used in a cellular context, it will generally be preferable to use an aptamer that has low affinity and/or high specificity for the components of the cell to be used.

As one of skill in the art will recognize upon reviewing this disclosure, the methods of the invention can be used with a wide variety of target molecules, and particularly with small molecules that are cell permeable. When a target molecule is not cell permeable, the target molecule can be applied to the host cell with an adjuvant, carrier, or other material that promotes cell permeabilization. Suitable agents include lipids, liposomes, polymers, and the like, including polycyclodextrin compounds.

A coding region is a polynucleotide region that, when present in a DNA form, can be expressed as an RNA molecule. The coding region can encode a polypeptide produced through translation of the RNA. The coding region can also encode an RNA that is itself functional in some way, such as an aptamer, a catalytic RNA, an RNA that regulates gene expression, etc. Certain coding regions are reporter regions, or regions encoding a reporter product. Certain coding regions are regulatory regions, or regions encoding a regulatory product. Certain coding regions are aptamer regions, or regions encoding an aptamer.

In view of this specification, one of skill in the art will be able to select a coding region encoding an appropriate reporter product. A reporter product is any detectable substance that is produced by transcription, and optionally translation, of a nucleic acid. Generally, reporter products are selected for ease of detection, as in the case of fluorescent proteins and proteins that degrade or produce fluorescent or chromogenic compounds. However, a variety of biomolecules are detectable by traditional antibody- or nucleic acid probe-based techniques, and therefore a large array of reporter products are available. For example, any gene for which the transcript can be detected by a technique such as Northern blotting or reverse transcriptase polymerase chain reaction may be used as a reporter gene. The presence or absence, and optionally the quantitative amount, of a reporter product may be observed by a variety of methods, including direct detection of the reporter product (e.g. detecting amounts of protein or nucleic acid) or detection of a product of the reporter product (e.g. detecting a result of enzymatic activity, emitted light, etc.). Exemplary reporter products include green fluorescent protein (GFP, and variants thereof, including red fluorescent protein, yellow fluorescent protein, etc., and variants that are optimized for different cell types, such as the eGFP protein optimized for use in eukaryotic cells) and beta-glucuronidase (encoded by the GUS gene). Toxins are a special class of reporter products. A toxin, as the term is used herein, can inhibit growth of a host cell or cause death of a host cell. The term “toxin” includes substances that have direct toxic effects as well as substances that have secondary or indirect toxic effects (e.g. enzymes that catalyze the production of further substances that have direct toxic effects). Toxins can be either polypeptides encoded by a coding region or RNA molecule encoded by a coding region. Suitable toxic proteins include, but are not limited to: barnase, colicins, cytolethal distending toxins, cytolysins, CcdB proteins (also known as “control of cell death”, “control of cell division” and “coupled cell division” proteins) and porins and mutants thereof. A toxin is preferably selected to be active on the host cell to be used. If the host cell is a bacterial cell, the toxin should be toxic to the bacterial cell, and if the host cell is a mammalian cell, the toxin should be toxic to the mammalian cell.

In view of this specification, one of skill in the art will be able to select a coding region encoding an appropriate regulatory product. In general, a regulatory product is any RNA or polypeptide that regulates the expression (e.g. transcription or translation) of another coding region. For example, one class of regulatory products are transcription factors. Transcription factors are proteins that regulate the transcription from a particular promoter. Transcription factors may be repressors (i.e. proteins that inhibit transcription) or activators (i.e. proteins that activate or enhance transcription). Transcription factors may also be regulated by phosphorylation, binding to an inducer, etc. An example of a regulatory product that is suitable for certain embodiments of the invention is the E. coli LacI protein. LacI is a transcriptional repressor that binds to the operator sites of the lac promoter and represses transcription. When LacI binds to galactose (or the membrane-permeable galactose analog isopropyl-beta-D-thiogalactopyranoside, “IPTG”), LacI no longer represses transcription, and the gene downstream of the lac promoter may be transcribed. Tetracycline transactivators and tetracycline transrepressors are transcriptional activators and repressors, respectively, that are well-suited for use in mammalian cells. These regulatory products are regulated by tetracycline. The T7 RNA polymerase is an example of a transcriptional activator that activates, and itself mediates, strong constitutive expression from a T7 promoter.

In certain embodiments, the invention provides nucleic acid constructs comprising an aptamer region (e.g. a region encoding an aptamer) and a coding region. As described above, the coding region may encode, for example, a reporter product or a regulatory product. The nucleic acid construct can be a single polynucleotide including DNA or RNA. When the nucleic acid construct is DNA, the nucleic acid construct can be introduced into the cell as either single stranded DNA or more preferably as double stranded DNA.

In certain embodiments, the invention provides a nucleic acid construct comprising an aptamer, that binds to a target molecule, operably linked to a coding region encoding a toxin (e.g. a toxic RNA or toxic polypeptide). In a preferred embodiment, the coding region encodes a toxic polypeptide. In certain preferred embodiments, the aptamer is positioned so as to inhibit production of the toxic product when it is bound to the target molecule. In this configuration, the construct may be used to generate cells that are dependent on the presence of the target molecule for growth or survival. Such cells may be used, for example, in selection systems to identify nucleic acids that encode gene products that are involved in the production of the target molecule. Such cells may also be used in biosensor systems. Although the target molecule may be essentially any compound, preferred target molecules are small molecules, and particularly small molecules that have poorly characterized biosynthetic pathways.

In certain embodiments, the invention provides a nucleic acid construct comprising an aptamer, that binds to a target molecule, operably linked to a coding region encoding a reporter product (i.e. any product the expression of which is readily detectable). In a preferred embodiment, the coding region encodes a reporter polypeptide, such as a fluorescent protein or a protein that produces a colored or fluorescent substrate. A reporter product may also be a toxic product, as cell death or growth cessation is a readily detectable phenomenon. In certain preferred embodiments, the aptamer is positioned so as to inhibit production of the reporter product when it is bound to the target molecule. In this configuration, the construct may be used to generate cells that are dependent on the presence of the target molecule for growth or survival. Such cells may be used, for example, in selection systems to identify nucleic acids that encode gene products that are involved in the production of the target molecule. Such cells may also be used in biosensor systems. Although the target molecule may be essentially any compound, preferred target molecules are small molecules, and particularly small molecules that have poorly characterized biosynthetic pathways. The nucleic acid construct may further comprise a promoter region. In certain embodiments, the promoter region is inducible such that, in certain configurations, the toxic product is only produced in the presence of the inducer (to activate the promoter) and the absence of the target molecule.

In certain embodiments, the invention provides systems of nucleic acid constructs. A system of nucleic acid constructs is any assemblage of two or more constructs that, when introduced into a cell, provide cross-regulation between the two or more constructs. For example, a system may have a first construct and a second construct. The first construct may comprise an aptamer and a coding sequence encoding a regulatory product. The second construct may comprise a promoter that is responsive to the regulatory product and a coding sequence that encodes a reporter product (including, for example, a toxin). This system of constructs may be designed using a regulatory product that is a repressor, such that the target molecule, by binding to the aptamer, inhibits production of the regulatory product; which in turn inhibits expression of the reporter product. The net effect of such a system is that the reporter product is produced only in the absence of the target molecule, and the expression of the reporter product is regulated by the aptamer, albeit indirectly. Such a system may be constructed by using, for example, a regulatory product that is a transcriptional repressor (e.g. the lacI repressor) of the promoter (e.g. the lac promoter) that drives expression of the reporter product. An alternative system may be formed by using a regulatory product that is a transcriptional activator of the promoter that drives expression of the reporter product. This alternate system will have the effect that the amount of reporter product produced decreases in the presence of the target molecule. Systems of nucleic acids may be designed with increasing complexity by stringing together a series of regulatory products and promoters, so long as the net effect remains that the formation of an aptamer:target molecule complex regulates expression of a reporter product. The various nucleic acid constructs involved in any system may be placed onto a single vector for delivery to a cell, or they may be delivered singly, either simultaneously or at different times.

Accordingly, using the concepts disclosed herein, one of skill in the art may assemble a system comprising an aptamer that binds to a target molecule and a nucleic acid encoding a reporter product, wherein the aptamer:target molecule regulates the production of the reporter product either positively or negatively, and either directly or indirectly. Likewise, one of skill in the art may use the concepts disclosed herein to generate a cell that comprises an aptamer and a reporter product, wherein an aptamer:target molecule complex regulates the production of the reporter product either positively or negatively, and either directly or indirectly.

In certain preferred embodiments, a nucleic acid construct of the invention further comprises a promoter region that is operably linked to a coding region. In certain embodiments, the promoter region is inducible. A number of promoter sequences are known to one of skill in the art and include for example, systems based on the lac operon that can be induced with IPTG and those based on the tet repressor such as those described by Schleif, R. (1992) in Transcriptional Regulation (CSHL Press, Cold Spring Harbor, NIA, pp. 643-665. The promoter may be positioned so as to drive expression of a single transcript comprising the aptamer and the coding region. As an example, one may generate a nucleic acid construct comprising a lac promoter that is positioned to drive expression of a transcript comprising an aptamer and a polypeptide toxin coding region. The aptamer inhibits translation of the toxin coding region. Therefore, in this example, if the toxin is produced when the inducer is present (galactose or IPTG, to activate the promoter) and the target molecule is absent (permitting translation of the toxin).

In certain embodiments, the nucleic acid construct is incorporated into a vector. Certain vectors have sequence information that allow the vectors to be propagated in a host cell. For example, a vector comprising a nucleic acid construct disclosed herein may be a plasmid for use with a bacterial cell. Such a plasmid will generally contain an origin of replication that allows it to be propagated within a bacterial host cell. A plasmid or other vector may also be designed to integrate into a chromosome of a host cell. The manipulation of plasmid or other vector DNA is well known to one of skill in the art (Sambrook, J.; Fritsch, E. F.; Maniatis, T. Molecular Cloning, A Laboratory Manual; 2nd ed.; Cold Spring Harbor Laboratory Press: 1989.). Other common vectors include viral vectors, containing at least some portion of a viral genome that assists in replication and/or integration of the vector in a host cell, and transposon vectors, containing at least some portion of a transposon (typically one or more terminal repeat sequences) that assists in replication and/or integration of a transposon in a host cell. In another embodiment, the nucleic acid construct is RNA and is introduced into the cell directly as RNA, using electroporation or a carrier, such as a lipid formulation, to introduce the nucleic acid into the host cell.

In certain embodiments, one or more nucleic acid of the invention is introduced into a host cell. A host cell is any cell capable of being cultured. Host cells are preferably bacteria.

Particularly preferred bacteria are E. coli, B. subtilis, Streptomyces antibioticus, Streptomyces mycarofaciens, Streptomyces avenmitilis, Streptomyces caelestis, Streptomyces tsukubaensis, Streptomyces fradiae, Streptomyces platensis, Streptomyces violaceoniger, Streptomyces ambofaciens, Streptomyces griseoplanus, and Streptomyces venezuelae. However, host cells may also be mammalian (e.g. CHO cells, fibroblasts, human embryonic kidney cells, adult or embryonic stem cells, hepatic cell lines, etc.), fungal (e.g. Saccharomyces cerevisiae), invertebrate (e.g. insect cells suitable for baculovirus-mediated gene expression, nematode cells) or plant cells. Nucleic acids may be introduced into host cells according to any method known in the art, including, for example, electroporation, tungsten particle bombardments (typically with plants and algae), calcium chloride mediated transformation, viral infection, lipofection, etc.

### 3. Methods for Nucleic Acid Cloning and Directed Evolution

In certain aspects, the invention relates to methods for identifying, or assisting in identifying, proteins that are involved in the metabolism, whether synthesis, degradation or other modification, of a target molecule. In certain aspects, the invention relates to methods for generating and identifying protein variants that have altered ability to synthesize, degrade or otherwise modify a target molecule.

A cell may be transformed with a nucleic acid construct or a plurality of nucleic acid constructs, at least one of which comprises an aptamer that interacts with a target molecule. The nucleic acid construct(s) are designed to create a system in the cell wherein the aptamer:target molecule complex regulates, directly or indirectly, the production of a reporter product. As described above, the system may comprise a single nucleic acid construct wherein the aptamer:target molecule complex directly regulates the production of the reporter product, or the system may comprise multiple nucleic acid constructs wherein the aptamer and the reporter product are not necessarily positioned in close proximity but, through the effects of intermediary regulatory products (e.g. transcription factors), the aptamer:target molecule complex nonetheless regulates the production of the reporter product.

A cell comprising the appropriate aptamer-reporter construct or system may be transformed with an exogenous nucleic acid construct (or a plurality of exogenous nucleic acid constructs) to generate a test cell. An exogenous nucleic acid construct is a nucleic acid molecule having a sequence contained therein that is foreign to the cell being used. The exogenous nucleic acid construct can be a library of nucleic acids in a common vector, for example, a cDNA library. A cDNA library can be derived from a particular organism or tissue. Depending on the application, a cDNA library can be selected that is likely to contain genes encoding the enzymatic activity of interest. For example, if a natural product is known to be produced in a particular organism, a cDNA library derived from the organism can be used.

Another suitable source of nucleic acid for use as an exogenous nucleic acid constructs is so called environmental DNA (eDNA). Handelsman, J.; Rondon, M. R; Brady, S. F.; Clardy, J.; Goodman, R. M. Chem. Biol. 1998, 5, R245-R249; Rondon, M. R. et al. Appl. Env. Microbiol. 2000, 66, 2541-2547; Brady, S. F.; Clardy, J. J. Am: Chem. Soc. 2000, 122, 12903-12904; Brady, S. F.; Chao, C. J.; Handelsman, J.; Clardy, J. Org. Lett 2001, 3, 1981-1984.

In yet another embodiment of the invention, the exogenous library is derived by directed evolution techniques such as random mutagenesis and gene shuffling. Using such techniques which are well known to one of skill in the art and are further described in U.S. Pat. Nos. 6,132,970; 5,605,793; 6,153,410; 6,177,263; see also, for example, Stemmer, “DNA Shuffling by Random Fragmentation and Reassembly: In Vitro Recombination for Molecular Evolution,” Proc. Natl. Acad. Sci., USA, vol. 91, October 1994, pp. 10747-10751; Kolkman J A, Stemmer W P C, “Directed evolution of proteins by exon shuffling” Nat. Biotechnol. 19: (5) 423-428, May 2001; Volkov A A, Arnold F H “Methods for in vitro DNA recombination and random chimeragenesis” in Applications Of Chimeric Genes And Hybrid Proteins, Pt C, Methods In Enzymology 328: 447-456, 2000. By examining the effects of this type of exogenous library, it is possible to identify modified forms of an enzyme (polypeptide variants) that are more effective at a particular task, such as biosynthesis or degradation of a target molecule.

The vector used to transform a test cell with an exogenous nucleic acid construct can be chosen to have characteristics suitable for the type of nucleic acid anticipated to be found. When a selection is developed using the methods of the invention for the synthesis of a complex natural product, multiple genes may be required for the synthesis. As such, the vector chosen preferably can contain segments of nucleic acid large enough to incorporate multiple genes, such as bacterial artificial chromosomes.

The effects of the exogenous nucleic acid on the amount of target molecule present in a cell may be monitored by observing the production of the reporter product. In embodiments employing a toxic reporter product that is turned off by the aptamer:target molecule complex, exogenous nucleic acids that increase the amount of target molecule may be efficiently identified by a cell viability selection. The terms “selection” and “screen” are used herein to refer to different processes (generally as the terms are used in the field of microbial genetics). A “selection” is a process whereby a population of cells (such as cells carrying different exogenous nucleic acid constructs) is exposed to a culture condition that is expected to kill (or stop the growth of) those cells that do not have a desired trait. A selection allows one of skill in the art to select for cells carrying the desired exogenous nucleic acid even when the exogenous nucleic acid is present in cells at an extremely low frequency (e.g. when fewer than one in one million, or even fewer than one in one billion cells carries an exogenous gene that increases the amount of target molecule in the cell). A screen is any method of identifying cells having a desired trait that does not involve a selection on the basis of cell survival. For example, when a reporter product is a fluorescent protein, cells having a desired property may be identified by screening a population of cells for cells having a fluorescence level that is predicted to correspond with a desired property. The detection of the relevant phenotype in a screen is generally made on a cell by cell basis (i.e. each cell is measured), meaning that a screen is far more labor intensive than a selection, where the vast majority of undesirable cells are eliminated in a single step.

In certain embodiments, it is desirable to identify an exogenous nucleic acid encoding an enzyme that catalyzes the biosynthesis of the target molecule from one or more precursor molecules. The test cell may be supplied with a source of the one or more precursor molecules (unless such molecules are normally present at sufficient levels in the cell), so that if the desired enzyme is produced in the cell, the target molecule is synthesized. If the test cell carries a construct or system in which the production of the reporter product decreases in response to the target molecule, then cells carrying an exogenous nucleic acid encoding a desirable enzyme may be identified by a decrease in the production of the reporter product. If the cell carries a construct or system in which the production of the reporter product increases in response to the target molecule, then cells carrying an exogenous nucleic acid encoding a desirable enzyme may be identified by an increase in the production of the reporter product. In this manner it is possible to identify representatives from a library of nucleic acids that encode enzymes that may be useful in the biosynthesis of the target molecule.

In certain embodiments, it is desirable to identify an exogenous nucleic acid encoding an enzyme that catalyzes the degradation of the target molecule. The test cell may be supplied with a source of the target molecule (unless such molecules are normally present at sufficient levels in the cell), so that if the desired enzyme is produced in the cell, the target molecule is degraded. If the cell carries a construct or system in which the production of the reporter product decreases in response to the target molecule, then cells carrying an exogenous nucleic acid encoding a desirable enzyme may be identified by an increase in the production of the reporter product. If the cell carries a construct or system in which the production of the reporter product increases in response to the target molecule, then cells carrying an exogenous nucleic acid encoding a desirable enzyme may be identified by a decrease in the production of the reporter product. In this manner it is possible to identify representatives from a library of nucleic acids that encode enzymes that may be useful in the degradation of the target molecule.

In certain embodiments, observing the effects of expression of the exogenous nucleic acid on the production of the reporter product may comprise a comparison to an appropriate control cell. The nature of the control cell will depend on the experimental design. For example, a control cell may comprise the same aptamer-reporter product system as the test cell, but lack the exogenous gene. Alternatively the control cell may comprise the same aptamer-reporter product system as the test cell, but fail to express the exogenous gene, perhaps because of a defective promoter or culture conditions that suppress expression. In embodiments where the test cell expresses a protein variant, the control cell may instead express a control protein. Typically a control protein will be a wild type or other previously characterized form of the protein from which the variants are derived. Observations in test cells may be compared to control cells cultured earlier, later or concurrently, or the observations may be compared to a standard level determined earlier, later or concurrently.

Exogenous nucleic acids or nucleic acids encoding protein variants identified by any of the methods described herein may be further characterized by, for example, purifying the expression product and testing the activity of that product with respect to the target molecule in vitro. For example, the ability of the expression product to bind, synthesize, degrade or modify the expression product may be assessed in vitro. In certain instances, further characterization will help identify and eliminate “false positive” clones.

### 4. Methods for Sensing the Presence or Absence of a Target Molecule

In certain embodiments, the invention provides methods for sensing the presence or absence of a target molecule by monitoring cell viability or growth. Accordingly, a cell-based biosensor may be constructed by introducing into a host cell a nucleic acid construct or a plurality of nucleic acid constructs, at least one of which comprises an aptamer that interacts with the target molecule to be sensed. The nucleic acid construct(s) are designed to create a system in the cell wherein the aptamer:target molecule complex regulates, directly or indirectly, the production of a toxin. Therefore, the production of the toxin provides a readout, or sensor, for the amount of aptamer:target molecule complex formed, and complex formation will generally be related to the concentration of target molecule that is present in the cell. In the case of cells designed such that the target molecule increases toxin production, cell death or decrease in growth will indicate the presence of the target molecule. In the case of cells designed such that the target molecule decreases toxin production, cell survival or growth will indicate the presence of the target molecule. Particularly in the case of cells that require the target molecule to survive or grow, it may be desirable to regulate expression of the toxin with a regulated promoter, so that toxin production can be suppressed at will even in the absence of the target molecule. This allows cells to be propagated and kept alive until they are to be used to sense the presence of the target molecule.

The cell-based biosensor may be used to sense the target molecule in essentially any context of interest. For example, cells that grow only in the presence of the target molecule may be distributed across a site, such as a landfill, a field, a soil sample, a water sample, an air sample etc. and the site can later be checked for the presence of the cells.

Cells may be encapsulated in a device that permits compounds from the environment to enter but does not permit the cells to leave. The device may then be placed at a site of interest and checked for alterations in cell growth.

Cells for use as a biosensor may be additionally transformed with a readily detectable marker that is continuously expressed (e.g. a fluorescent protein), so that the size of a cell population, and its growth or decline, may be assessed easily.

### Exemplification

The invention now being generally described, it will be more readily understood by reference to the following examples, which are included merely for purposes of illustration of certain aspects and embodiments of the present invention, and are not intended to limit the invention.

### EXAMPLE 1

### Cloning a Theobromine N-Methyltransferase

A nucleic acid construct is generated having an aptamer sequence that interacts with caffeine but not the caffeine precursor, theobromine. A nucleic acid sequence for a similar aptamer is described in Jenison, R. D.; Gill, S. C.; Pardi, A.; Polisky, B. Science 1994, 263, 1425-1429 that can distinguish caffeine from the related theophylline. Using the techniques described Complementary strands of a DNA molecule encoding the aptamer sequence is synthesized on a DNA synthesizer using standard phosphoramidite chemistry. The aptamer sequence is ligated to a plasmid having an inducible promoter and a toxic polypeptide coding region. The plasmid also contains a gene for the resistance to the antibiotic ampicillin.

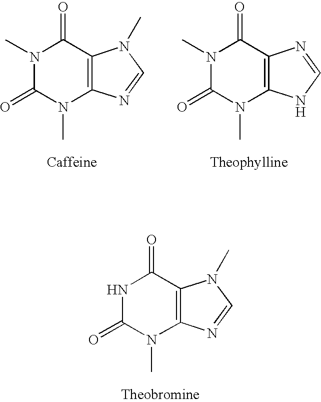

The aptamer construct is transformed into E. coli, and the transformed cells are grown in broth containing ampicillin. The transformed cells are then transformed with a cDNA library derived from coffee plants having a kanamycin resistance gene in the plasmid vector. The doubly transformed cells are grown in broth containing both ampicillin and kanamycin. The cells are grown in the presence of theobromine. Expression of the aptamer-toxin. construct is induced by adding IPTG to the broth and the cells are plated on agar plates containing both kanamycin and ampicillin. The colonies are allowed to grow overnight at 37 degrees C. in an incubator, and colonies are picked. Plasmid DNA is isolated from the growing colonies. The nucleic acid sequence in the plasmid DNA containing coffee plant DNA is isolated and sequenced to determine the identity of the gene responsible for the conversion of theobromine into caffeine.

### EXAMPLE 2

### Cloning a Caffeine N-Demethylase

A nucleic acid construct is generated having an aptamer sequence that interacts well with theophylline but poorly with the theophylline precursor, caffeine. A nucleic acid sequence for a similar aptamer is described in Jenison, R. D.; Gill, S. C.; Pardi, A.; Polisky, B. Science 1994, 263, 1425-1429 that can distinguish caffeine from the related theophylline. Using the techniques described, complementary strands of a DNA molecule encoding the aptamer sequence is synthesized on a DNA synthesizer using standard phosphoramidite chemistry. The aptamer sequence is ligated to a plasmid having an inducible promoter and a toxic polypeptide coding region. The plasmid also contains a gene for the resistance to the antibiotic ampicillin.

The aptamer construct is transformed into E. coli, and the transformed cells are grown in broth containing ampicillin. The transformed cells are then transformed with a cDNA library derived from coffee plants having a kanamycin resistance gene in the plasmid vector. The doubly transformed cells are grown in broth containing both ampicillin and kanamycin. The cells are grown in the presence of caffeine. Expression of the aptamer-toxin. construct is induced by adding IPTG to the broth and the cells are plated on agar plates containing both kanamycin and ampicillin. The colonies are allowed to grow overnight at 37 degrees C. in an incubator, and colonies are picked. Plasmid DNA is isolated from the growing colonies. The nucleic acid sequence in the plasmid DNA containing coffee plant DNA is isolated and sequenced to determine the identity of the gene responsible for the conversion of caffeine into theophylline.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A nucleic acid construct comprising:
a) a first coding region encoding an aptamer that interacts with a target molecule to form an aptamer:target molecule complex; and
b) a second coding region encoding a toxin,
wherein the production of the toxin is inhibited by the aptamer:target molecule complex.

2-18. (canceled)

19. A cell comprising:
a) a nucleic acid encoding an aptamer that binds to a target molecule to form an aptamer:target molecule; and
b) a nucleic acid encoding a toxin;
wherein the production of the toxin is regulated by the aptamer:target molecule complex.

20. The cell of claim 19, wherein the aptamer is positioned to directly regulate the production of the toxin.

21. The cell of claim 19, wherein the aptamer indirectly regulates the production of the toxin.

22. The cell of claim 21, wherein the aptamer is positioned so as to directly regulate the expression of a coding sequence encoding a regulatory product, and wherein the regulatory product regulates production of the toxin.

23. The cell of claim 21, wherein the nucleic acid encoding the aptamer and the nucleic acid encoding a toxin are present in a single vector.

24. The cell of claim 21, wherein the nucleic acid encoding the aptamer and the nucleic acid encoding a toxin are present in separate vectors.

25. A method for cloning or assisting in cloning a nucleic acid encoding a product involved in the metabolism of a target molecule, the method comprising:
a) culturing a test cell comprising:
i) an exogenous nucleic acid;
ii) a coding region encoding an aptamer that binds a target molecule to form an aptamer:target molecule complex; and
iii) a coding region encoding a reporter product,
wherein the aptamer:target molecule complex regulates production of the reporter product;
b) observing an effect of the expression of the exogenous nucleic acid on the production of the reporter product, wherein an exogenous nucleic acid that affects the production of the reporter product is nucleic acid encoding a product involved in the metabolism of a target molecule.

26. The method of claim 25, wherein the expression of the exogenous nucleic acid decreases the production of the reporter product.

27. The method of claim 25, wherein the expression of the exogenous nucleic acid increases the production of the reporter product.

28. The method of claim 26, wherein the reporter product is a toxin.

29. The method of claim 25, wherein the reporter product is selected from the group consisting of: a fluorescent protein, an enzyme that modifies a detectable substrate and an enzyme that catalyzes the production of a detectable product.

30. The method of claim 25, wherein the exogenous nucleic acid is operably linked to a conditional promoter.

31. The method of claim 25, wherein the exogenous nucleic acid is a representative nucleic acid from a nucleic acid library.

32. The method of claim 25, wherein the coding region encoding an aptamer and the coding region encoding a reporter product are expressed as a single transcript.

33. The method of claim 25, wherein the test cell is selected from the group consisting of: a bacterial cell, a fungal cell, a plant cell and a vertebrate cell.

34. The method of claim 25, wherein the test cell is an E. coli cell.

35. The method of claim 25, wherein observing an effect of the expression of the exogenous nucleic acid on the production of the reporter product comprises comparing the production of the reporter product in the test cell to the production of the reporter product in an appropriate control cell.

36. The method of claim 35, wherein the appropriate control cell is a cell substantially identical to the test cell but cultured in conditions that inhibit expression of the exogenous nucleic acid.

37. The method of claim 35, wherein the appropriate control cell is a cell substantially identical to the test cell but lacking the exogenous nucleic acid.

38. A method for identifying, or assisting in identifying, a protein variant having an altered activity with respect to a target molecule, the method comprising:
a) culturing a test cell comprising:
i) a nucleic acid encoding a protein variant;
ii) a coding region encoding an aptamer that binds a target molecule to form an aptamer:target molecule complex; and
iii) a coding region encoding a reporter product,
wherein the aptamer:target molecule complex regulates production of the reporter product;
b) observing an effect of the expression of the nucleic acid encoding the protein variant on the production of the reporter product; and
c) comparing the effect of the expression of the nucleic acid encoding the protein variant to the effect of expression of a control protein,
wherein a nucleic acid encoding a protein variant that alters the production of the reporter product as compared to the control protein is a nucleic acid encoding a protein variant with an altered activity with respect to the target molecule.

39. The method of claim 38, wherein altering the sequence encoding the protein comprises making an alteration selected from the group consisting of: an insertion mutation, a deletion mutation, a point mutation, an exon shuffle and a mixture of the foregoing alterations.

40. The method of claim 38, wherein the altered activity with respect to the target molecule is an altered ability to catalyze the synthesis of the target molecule.

41. The method of claim 38, wherein the altered activity with respect to the target molecule is an altered ability to catalyze the degradation of the target molecule.

42. A method for detecting the presence of a target molecule, the method comprising:
a) culturing a cell in an environment suspected of containing the target molecule, the cell comprising:
i) a coding region encoding an aptamer that binds a target molecule to form an aptamer:target molecule complex; and
ii) a coding region encoding a toxin,
wherein the aptamer:target molecule complex regulates production of the toxin;
b) observing the production of the toxin after placing the cell in the environment suspected of containing the target molecule, wherein an environment that causes an alteration in the production of the toxin is an environment that contains the target molecule.

43. The method of claim 42, wherein observing the production of the toxin comprises observing cell death and/or cessation of cell growth.

44. The method of claim 42, wherein the aptamer:target molecule complex inhibits toxin production, and wherein an environment that permits cell growth is an environment that contains the target molecule.
